(19) United States
(12) Patent Application Publication
(10) Pub. No.: US 2014/0051888 A1
(43) Pub. Date: Feb. 20, 2014

(54) NOVEL STEREOISOMERIC MIXTURES, SYNTHESIS AND USES THEREOF

(71) Applicant: AMPAC FINE CHEMICALS LLC, Rancho Cordova, CA (US)

(72) Inventors: William DUBAY, Folsom, CA (US); Jeffrey D. BUTLER, Folsom, CA (US); Charles L. LIOTTA, Atlanta, GA (US)

(73) Assignee: AMPAC FINE CHEMICALS LLC, Rancho Cordova, CA (US)

(21) Appl. No.: 13/955,086

(22) Filed: Jul. 31, 2013

Related U.S. Application Data
(60) Provisional application No. 61/691,031, filed on Aug. 20, 2012.

(51) Int. Cl. C07C 209/88

(52) U.S. Cl. CPC C07C 209/88
USPC 564/302

(57) ABSTRACT

A novel stereochemical mixture of 1,6-diaryl-2,5-diaminohexanes, such as a mixture of stereoisomers of 1,6-diphenylhexane-2,5-diamine, is described. Also described are methods of preparing stereochemically pure 1,6-diaryl-2,5-diaminohexanes, and particularly stereochemically pure 1,6-diphenyl-2,5-diaminohexane. Also described is the use of both the mixture of stereoisomers and the individual stereoisomers.

### CROSS REFERENCE TO RELATED APPLICATIONS

This application is entitled to priority pursuant to 35 U.S.C. §119(e) to U.S. Provisional Application No. 61/691,031, filed on Aug. 20, 2012, the content of which is hereby incorporated by reference in its entirety.

### FIELD OF THE INVENTION

Embodiments of the present invention relate to (1) novel stereoisomeric mixtures of 1,6-diaryl-2,5-diaminohexanes, (2) the preparation of such mixtures, and (3) methods of preparing stereoisomerically pure 1,6-diaryl-2,5-diaminohexanes from the mixtures of stereoisomers.

### BACKGROUND OF THE INVENTION

Enantiomerically pure (2R,5R)-1,6-diphenyl-2,5-diaminohexane is used as an intermediate in the synthesis of pharmaceutically active ingredients, such as cobicistat. The current method for production of enantiomerically pure (2R,5R)-1,6-diphenyl-2,5-diaminohexane relies, in part, on a step involving a chiral aziridine dimerization to construct the diamine core. In addition, an N,N-dimethylsulfamoyl protecting group is a critical component of the synthesis. The use of a chiral aziridine presents several problematic factors. Aziridines are strained three-membered rings and, as such, are highly reactive. As a consequence, in addition to safety issues, unwanted side products usually accompany their use. In addition, chiral aziridines are expensive and employing them in a commercial process adds substantially to the cost of the process.

Therefore, there is a need for more facile and cost-effective production of enantiomerically pure (2R,5R)-1,6-diphenyl-2,5-diaminohexane. In addition, there is a need for more facile and cost-effective synthesis of a wide variety of 1,6-diaryl-2,5-diaminohexanes both as a mixture of stereoisomers and as stereoisomerically pure compounds. Such needs are met by the present invention.

### BRIEF SUMMARY OF THE INVENTION

The invention herein relates to a novel synthetic route to provide a mixture of stereoisomers of 1,6-diaryl-2,5-diaminohexanes by subjecting a mixture of stereoisomers of 2,5-diarylmethylenehexanedioic acids to conditions promoting a bis-decarboxylative bis-amination reaction. The resulting mixture of stereoisomers of the 1,6-diaryl-2,5-diaminohexanes can then be used to prepare the stereochemically pure (2R,5R)-, (2S,5S)-, or meso-1,6-diaryl-2,5-diaminohexanes. The method provides cost and processing advantages, as compared to current methods of synthesis. It increases the availability of 1,6-diarylhexane-2,5-diamines for use as a reagent, e.g., starting material, thereby broadening practical avenues of synthesis for active pharmaceutical ingredients.

In one general aspect, the present invention relates to a method of producing a mixture of stereoisomers of formula (I):

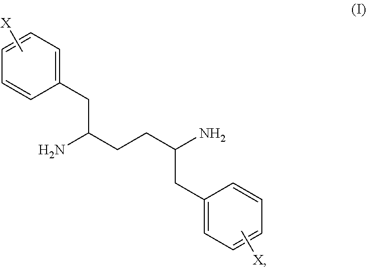

or pharmaceutically acceptable salts thereof,
comprising subjecting a stereoisomeric mixture of formula (II):

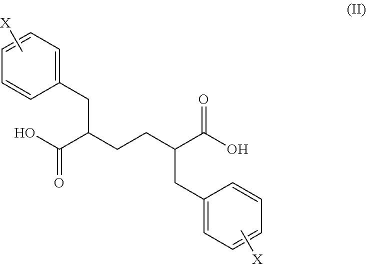

or pharmaceutically acceptable salts thereof to a bis-decarboxylative bis-amination reaction, wherein the mixture of stereoisomers of formula (I) comprises (2R,5R)-, (2S,5S)- and meso configurations of formula (I), and X is independently selected from the group consisting of hydrogen, halogen, cyano, nitro, alkyl, aryl, alkoxy, polyfluoroalkyl and polyfluoroaryl.

In a preferred embodiment, the bis-decarboxylative bis-amination reaction comprises a Curtius rearrangement.

In another general aspect, the present invention relates to a method of preparing a stereoisomerically pure compound of formula (Ia) of the (2R,5R) configuration:

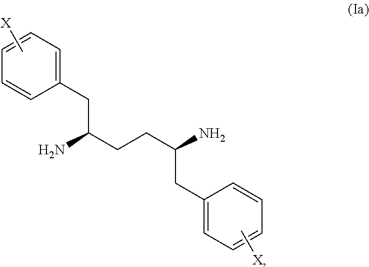

or a pharmaceutically acceptable salt thereof;

a stereoisomerically pure compound of formula (1b) of the (2S,5S) configuration:

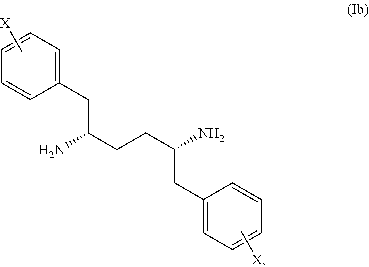

or a pharmaceutically acceptable salt thereof; or

a stereoisomerically pure compound of formula (Ic) of the meso configuration:

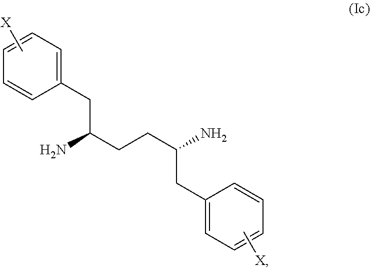

or a pharmaceutically acceptable salt thereof; the method comprising:

(i) producing a mixture of stereoisomers of formula (I):

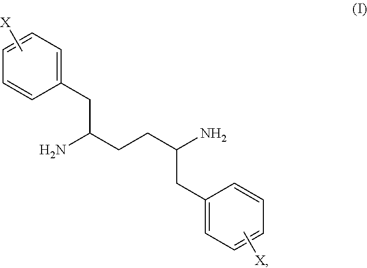

or pharmaceutically acceptable salts thereof, wherein the mixture comprises (2R,5R)-, (2S,5S)-, and meso configurations of formula (I), and X is independently selected from the group consisting of hydrogen, halogen, cyano, nitro, alkyl, aryl, alkoxy, polyfluoroalkyl and polyfluoroaryl; and

(ii) isolating the stereoisomerically pure compound of formula (Ia), (Ib), or (Ic), or the pharmaceutically acceptable salt thereof from the mixture of stereoisomers.

In a preferred embodiment, the stereochemically pure compound of formula (Ia), (Ib), or (Ic) is isolated from a mixture of stereoisomers of formula (I) using a chromatographic technique, and preferably a continuous chromatographic technique such as a simulated moving bed (SMB) chromatography.

According to an embodiment of the present invention, a method of preparing a stereoisomerically pure (2R,5R)-1,6-diphenyl-2,5-diaminohexane:

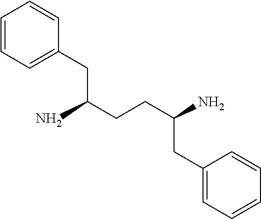 [SMILES: N[C@H](CC[C@@H](N)Cc1ccccc1)Cc1ccccc1]

or a pharmaceutically acceptable salt thereof, comprises:

(i) producing a mixture of stereoisomers of 1,6-diphenyl-2,5-diaminohexane:

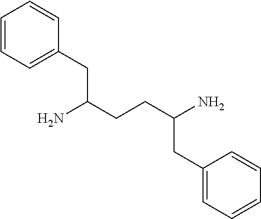 [SMILES: NC(CCC(N)Cc1ccccc1)Cc1ccccc1]

or pharmaceutically acceptable salts thereof, wherein the mixture comprises (2R,5R)-1,6-diphenyl-2,5-diaminohexane, (2S,5S)-1,6-diphenyl-2,5-diaminohexane, and meso-1,6-diphenyl-2,5-diaminohexane; and

(ii) isolating the stereoisomerically pure (2R,5R)-1,6-diphenyl-2,5-diaminohexane or the pharmaceutically acceptable salt thereof from the mixture by a method comprising a chromatographic technique.

In yet another general aspect, the present invention relates to a mixture of stereoisomers of formula (I):

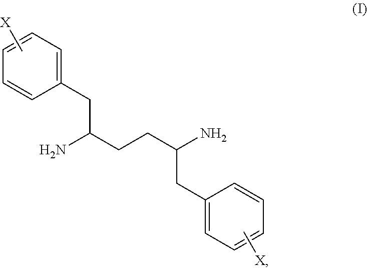

or pharmaceutically acceptable salts thereof. The mixture comprises (2R,5R)-, (2S,5S)-, and meso configurations of formula (I), and X is independently selected from the group consisting of hydrogen, halogen, cyano, nitro, alkyl, aryl, alkoxy, polyfluoroalkyl and polyfluoroaryl.

In a preferred embodiment, the mixture comprises (2R,5R)-1,6-diphenyl-2,5-diaminohexane, (2S,5S)-1,6-diphenyl-2,5-diaminohexane, and meso-1,6-diphenyl-2,5-diaminohexane.

Other aspects, features and advantages of the invention will be apparent from the following disclosure, including the detailed description of the invention and its preferred embodiments and appended claims.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

The phrase “pharmaceutically acceptable salt” as used herein means those salts of a compound of interest that are safe and effective for administration to a mammal and that possess the desired biological activity. Pharmaceutically acceptable acid addition salts include, but are not limited to hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds used in the present invention can form pharmaceutically acceptable salts with various amino acids. Suitable base salts include, but are not limited to, aluminum, calcium, lithium, magnesium, potassium, sodium, zinc, bismuth, and diethanolamine salts. For a review of pharmaceutically acceptable salts see Berge et al., J. Pharm. Sci. (1977) 66, 1-19, incorporated herein by reference.

Methods for preparing salts of a particular stereoisomer of a compound, or for preparing salts of mixtures of stereoisomers of a compound are well known to one skilled in the art. For example, a hydrochloride salt can be produced by reacting a compound produced by a method of the present invention with HCl in an organic solvent, such as dioxane or other suitable solvents. Methods for preparing other salts in addition to the hydrochloride salt will be well known to one of ordinary skill in the art.

In a chiral compound, carbon atoms are often, but not exclusively, at the chiral or stereogenic center. As used herein, a “stereogenic or chiral center” refers to a carbon atom to which four different atoms or groups of atoms are attached. For example, the compound 1,6-diphenylhexane-2,5-diamine has two chiral centers, one at carbon 2 and one at carbon 5. Each of these chiral centers is attached to four different atoms or groups of atoms, with one being an amine functionality.

As used herein, the term “stereoisomers” refers to at least two compounds having the same molecular formula and connectivity of atoms, but having a different arrangement of atoms in a three-dimensional space. In view of the present disclosure, a stereoisomer can be, for example, an enantiomer, a diastereomer, or a meso compound.

The “RS” convention for designating the three dimensional arrangement of atoms at a chiral or stereogenic center will be employed herein in the naming of stereoisomers. The designation of “S” or “R” for a given chiral or stereogenic center is based upon “priority rules” well known to those skilled in the art.

As used herein, the term “enantiomers” refers to a pair of compounds which are non-superimposable mirror images of one another. In other words, an “enantiomer” is a stereoisomer that cannot be superimposed on its mirror image. Chemists employ various naming conventions to distinguish enantiomers from one another. Because an enantiomer can rotate plane-polarized light, chemists sometime designate enantiomers using the symbols (+) and (−) or d and l depending on whether they rotate plane-polarized light in a clockwise or counterclockwise direction, respectively. The former enantiomer is termed to be dextrorotatory and the latter enantiomer is termed to be levorotatory. As a result of this behavior in the presence of plane-polarized light, enantiomers have also been referred to as “optical isomers.” Enantiomers have identical physical and chemical properties in an achiral environment but each rotates the plane of polarized light to the same number of degrees but in the opposite direction.

As used herein, the term “optical rotation” refers to the number of degrees of rotation of plane polarized light exhibited by an optically active stereoisomer, such as an enantiomer, either neat or in solution. Optical rotation is usually measured with a polarimeter. The “specific rotation” is calculated from the observed optical rotation taking into account the concentration of the optically active molecule and the dimensions of the vessel containing the optically active molecules. The wavelength of plane polarized light usually employed is the “D line” of sodium.

1,6-diaryl-2,5-diaminohexanes of formula (I) have one enantiomeric pair of molecules. Their names, employing the stereochemical priority rules, are (2R,5R)-1,6-diaryl-2,5-diaminohexane (formula (Ia)) and (2S,5S)-1,6-diaryl-2,5-diaminohexane (formula (Ib)):

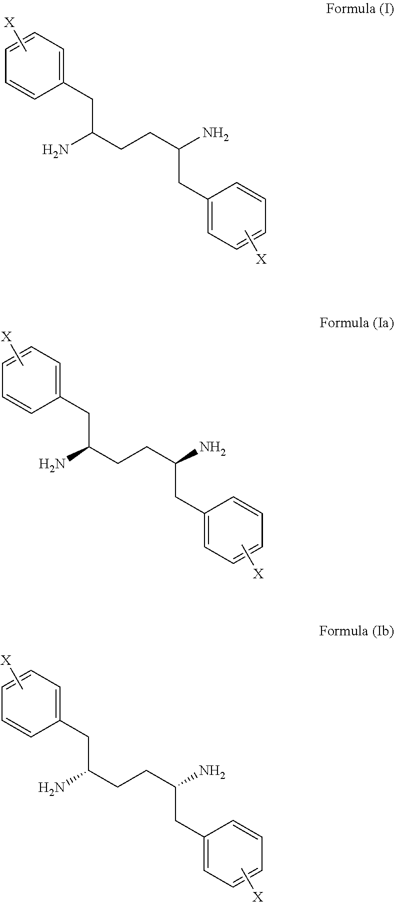

As used herein, a “meso compound” is a stereoisomer having at least two chiral or stereogenic centers, but which also possesses an internal plane of symmetry such that the compound is optically inactive (i.e., does not rotate the plane of polarized light). For example, one of the stereoisomers of a 1,6-diaryl-2,5-diaminohexane compound of formula (I) has the meso stereoconfiguration of formula (Ic):

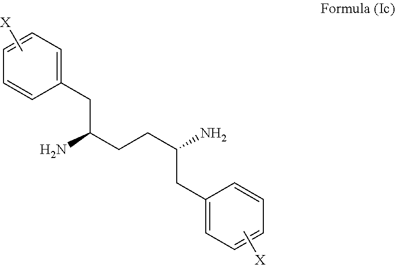

This meso stereoisomer of formula (Ic) has one chiral or stereogenic center of the S configuration and one chiral or stereogenic center of the R configuration. (2R,5S)-1,6-diaryl-2,5-diaminohexane is identical to (2S,5R)-1,6-diaryl-2,5-diaminohexane. The meso configuration of 1,6-diaryl-2,5-diaminohexane is optically inactive.

As used herein, “diasteromers” are stereoisomers which are not mirror images. For example, (2R,5R)-1,6-diaryl-2,5-diaminohexane and meso-1,6-diaryl-2,5-diaminohexane are diastereomers. (2S,5S)-1,6-diaryl-2,5-diaminohexane and meso-1,6-diaryl-2,5-diaminohexane are also diastereomers. Diastereomers have different chemical and physical properties in both an achiral environment and in a chiral environment.

As used herein, the terms “stereochemically pure” and “stereoisomerically pure” refer to a composition or compound containing a substantially pure stereoisomer of a compound. The term “substantially pure,” when used with reference to a stereoisomer, such as an enantiomer, diastereomer, or meso compound, means that the composition or compound is substantially free of all the other stereoisomers of that compound, but not necessarily free from other materials (e.g., solvents, other compounds, etc.). For example, a stereochemically pure 1,6-diphenylhexane-2,5-diamine can be a substantially pure (2S,5S)-1,6-diphenylhexane-2,5-diamine, a substantially pure (2R,5R)-1,6-diphenylhexane-2,5-diamine, or a substantially pure meso-1,6-diphenylhexane-2,5-diamine.

According to embodiments of the present invention, a stereochemically pure composition or compound comprises about 97% by weight (w/w) or greater, such as 97%, 97.5%, 98%, 98.5%, 99%, 99.5%, 99.8% or 100% (w/w) of a single stereoisomer of a particular compound relative to the total weight of all the stereoisomers of the compound.

A stereoisomerically pure composition or compound can be “enantiomerically pure” or “optically pure,” which refers to a composition containing a substantially optically pure enantiomer. As used herein, “substantially optically pure,” when used with reference to a stereoisomer of a compound that rotates the plane of polarized light, such as an enantiomer or diastereomer, means that the composition or compound is substantially free of the other stereoisomers of that compound, but not necessarily free from other materials, such that the optical rotation of the composition or compound is substantially equal to the specific rotation of the stereoisomer. For example, an enantiomerically pure 1,6-diphenylhexane-2,5-diamine can be a substantially optically pure (2S,5S)-1,6-diphenylhexane-2,5-diamine or a substantially optically pure (2R,5R)-1,6-diphenylhexane-2,5-diamine.

As used herein, the terms “stereoisomeric mixture” and “mixture of stereoisomers” refer to a mixture or composition containing at least two stereoisomers of a particular compound. A stereoisomeric mixture can comprise all possible stereoisomers of a particular compound, including all enantiomers, all diastereomers, and meso compounds. A stereoisomeric mixture can also comprise only two stereoisomers of a particular compound, and can be, for example, a mixture of two enantiomers, or a mixture of an enantiomer and a diastereomer. The amounts of each stereoisomer of a compound present in a stereoisomeric mixture can be equal or unequal. For example, if two stereoisomers are present in a stereoisomeric mixture, the mixture can contain each of the two stereoisomers at about 50% of the mixture relative to a total of 100%, or the mixture can contain one stereoisomer at about 75% of the mixture and the other 25% of the mixture, relative to a total of 100%.

According to embodiments of the present invention, a stereoisomeric mixture of 1,6-diaryl-2,5-diaminohexanes comprises the (2R,5R), (2S,5S) and meso configurations of 1,6-diaryl-2,5-diaminohexane. The amount of each stereoisomer present in the stereoisomeric mixture can vary independently, for example, from 0.01% to 99.98% (w/w), such that a total of (2R,5R), (2S,5S), and meso configurations in the mixture is 100% (w/w). For example, a ratio of (2R,5R):(2S,5S):meso configurations in a stereoisomeric mixture of 1,6-diaryl-2,5-diaminohexanes can be 40%:40%:20% (i.e. unequal). The ratio can also be 33.3%:33.3%:33.3% (i.e. equal). In a preferred embodiment, the ratio is 25%:25%:50%. The above are intended to be illustrative of particular embodiments of the present invention, and are not intended to be limiting in any way.

According to embodiments of the present invention, a mixture of stereoisomers can be a racemic mixture. As used herein, a “racemic mixture” or a “racemate” refers to a composition containing a mixture of equal amounts of enantiomers (50/50). Although each individual enantiomer present in the racemate is optically active, the mixture itself is not optically active. For example, a racemic mixture of 1,6-diphenylhexane-2,5-diamine comprises a mixture of equal amounts of (2S,5S)-1,6-diphenylhexane-2,5-diamine and (2R,5R)-1,6-diphenylhexane-2,5-diamine. Although (2S,5S)-1,6-diphenylhexane-2,5-diamine is optically active, and (2R,5R)-1,6-diphenylhexane-2,5-diamine is optically active, a 50:50 mixture of the (2S,5S) and (2R,5R) configurations of 1,6-diphenylhexane-2,5-diamine is not optically active. According to an embodiment of the present invention, a racemic mixture can be obtained by purifying a stereoisomeric mixture comprising (2R,5R)-, (2S,5S)-, and meso-1,6-diaryl-2,5-diaminohexane according to the invention to separate the meso-compound from the enantiomeric pair by, for example, chromatography.

As used herein, “decarboxylative amination” is defined as the process of substituting an amino functionality or a derivative of an amino functionality for a carboxyl group, a carboxylate group, or a derivative of a carboxyl group. Derivatives of a carboxyl group and an amino functionality are well known to those skilled in the art. Examples of derivatives of a carboxyl group include, but are not limited to, amides, hydroxamic acids, esters, acyl halides, and acyl azides. Examples of derivatives of an amino functionality include, but are not limited to, carbamate derivatives, such as tert-butyl carbamates (Boc) and the like. A decarboxylative amination reaction can proceed, for example, through a Curtius rearrangement of an acylazide, a Hoffmann rearrangement of an amide, or a Lossen rearrangement of a hydroxamic acid or a hydroxamate derivative.

As used herein, “bis-decarboxylative bis-amination” is defined as the process of substituting amino functionalities or derivatives of amino functionalities for two carboxyl groups, two carboxylate groups, or derivatives of two carboxyl groups.

Unless otherwise noted, the term “alkyl” as used herein means a saturated, unbranched or branched hydrocarbon chain. An alkyl group can be unsubstituted or substituted with one or more suitable substituents. Examples of unbranched alkyls include, but are not limited to methyl, ethyl, propyl, butyl, pentyl, hexyl, heptyl, nonyl, and decyl. Examples of branched alkyls include, but are not limited to isopropyl, isobutyl, 2-methylpentane, 3-methylpentane, 2,2-dimethylbutane, 2,3-dimethylbutane, 2-methylhexane, 2,3-dimethylhexane, 2,2-dimethylhexane, and 3,3-dimethylhexane.

Unless otherwise noted, the term “alkoxy” as used herein, denotes a unit having the general formula —OR wherein R is an alkyl, aryl, alkenyl, or alkynyl unit. An alkoxy group can be, for example, methoxy, ethenyloxy, and ethynyloxy. Other examples of alkoxy groups include, but are not limited to, ethoxy, propoxy, butoxy, isobutoxy, sec-butoxy, t-butoxy, hexyloxy, and the like. An alkoxy group can be unsubstituted or substituted with one or more suitable substituents. Unless otherwise noted, the term “aryloxy” as used herein, denotes a unit such as phenoxy and substituted phenoxy groups.

As used herein, the term “aryl” is intended to mean any substituent derived from an aromatic ring. An aryl substituent can be a single aromatic ring, or multiple aromatic rings. Examples of aryl groups include, but are not limited to, phenyl, napthyl, methylbenzyl, and dimethylbenzyl. An aryl group can also be a heteroaryl group, wherein one or more carbon atoms of an aromatic ring is substituted with a heteroatom, such as oxygen, nitrogen or sulfur. Examples of heteroaryl substituents include, but are not limited to pyridyl, furanyl, and thienyl. Aryl and heteroaryl groups can be unsubstituted or substituted with one or more suitable substituents.

When a particular group is “substituted” (i.e. alkyl, aryl, heteroaryl, or alkoxy), that group can have one or more substituents preferably from one to five substituents, more preferably from one to three substituents, most preferably from one to two substituents, independently selected from the list of substituents. Representative examples of suitable substituents for which a particular group can be substituted with include halogens, such as fluorine, chlorine, bromine, and iodine; alkyl, such as methyl, ethyl, and propyl; alkenyl, such as ethenyl, propenyl, and butenyl; alkynl, such as ethynyl, propynyl, and butynyl; aryl or heteroaryl, such as phenyl and pyridyl; alkoxy, such as methoxy, ethoxy, and propoxy; nitriles, such as cyano; and nitro. For example, a substituted aryl substituent can be a polyfluoroaryl group, meaning that the substituent is an aryl group, such as phenyl, substituted with one or more fluorine atoms, such as, e.g., 2,3-difluorophenyl. As another illustrative example, a substituted alkyl substituent can be a polyfluoroalkyl group, meaning that the substituent is an alkyl group, such as ethyl, substituted with one or more fluorine atoms, such as, e.g., 2,2-difluoroethyl.

As used herein, the terms “para,” “meta,” and “ortho” refer to a location of substitution on a substituted aryl group. An ortho-substituted aryl group has at least two substituents that are attached to adjacent carbons and occupy positions next to one another (i.e. at carbons 1 and 2), and can be referred to as a 1,2-substitution pattern. A meta-substituted aryl group has at least two substituents that are attached at carbons 1 and 3, and can be referred to as a 1,3-substitution pattern. A para-substituted aryl ring has at least two substituents that are attached at carbons 1 and 4, and can be referred to as a 1,4-substitution pattern.

As used herein, the term “halogen” means fluorine, chlorine, bromine, or iodine. Correspondingly, the term “halo” means fluoro, chloro, bromo, or iodo.

In one general aspect, the present invention relates to a method of producing a mixture of stereoisomers of the formula (I):

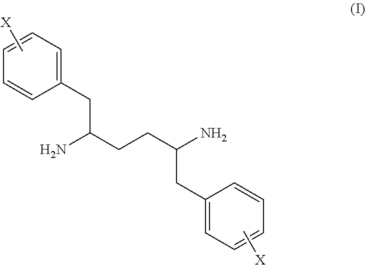

or pharmaceutically acceptable salts thereof, the method comprising subjecting a stereoisomeric mixture of formula (II):

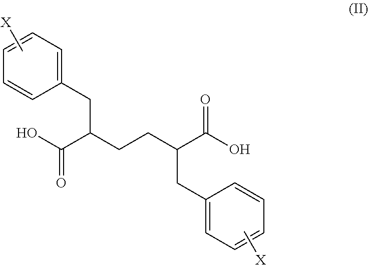

or pharmaceutically acceptable salts thereof, to a bis-decarboxylative bis-amination reaction, wherein the mixture of stereoisomers of formula (I) comprises (2R,5R)-, (2S,5S)-, and meso configurations of formula (I). According to embodiments of the present invention, each of the aryl groups of formula (I) is optionally substituted with a substituent X selected from the group consisting of halogen, cyano, nitro, alkyl, aryl, alkoxy, polyfluoroalkyl and polyfluoroaryl, preferably, the X substituents for the aryl groups are identical and are located at the identical para-, meta-, or ortho-positions in the aryl groups.

As an illustrative non-limiting example of the present invention, a mixture of stereoisomers of formula (I) can be a mixture of stereoisomers of 1,6-(di-4-fluorophenyl)-2,5-diaminohexane, wherein each aryl ring is para-substituted with a fluorine atom (i.e. X is fluorine) as follows:

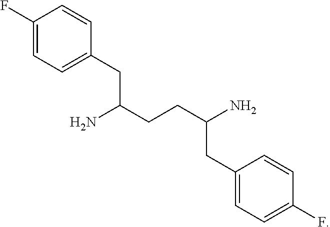 [SMILES: NC(CCC(N)Cc1ccc(F)cc1)Cc1ccc(F)cc1]

According to a preferred embodiment of the present invention, each of the aryl rings of a stereoisomer of formula (I) is unsubstituted, such that a mixture of stereoisomers of formula (I) contains (2R,5R)-, (2S,5S)-, and meso 1,6-diphenyl-2,5-diaminohexane. 1,6-diphenyl-2,5-diaminohexane has the following chemical structure:

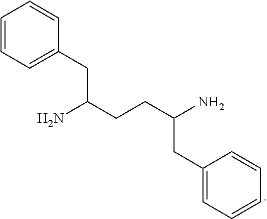 [SMILES: NC(CCC(N)Cc1ccccc1)Cc1ccccc1]

According to embodiments of the present invention, the bis-decarboxylative bis-amination reaction used in the present invention comprises one selected from the group consisting of a Curtius rearrangement, a Hoffmann rearrangement and a Lossen rearrangement. Examples of such bis-decarboxylative bis-amination reactions include, but are not limited to, a Curtius rearrangement of a diacyl azide, a Hoffmann rearrangement of a diamide, or a Lossen rearrangement of a dihydroxamic acid or a dihydroxamic acid derivative.

Preferably, the bis-decarboxylative bis-amination reaction comprises a Curtius rearrangement.

In one embodiment of the present invention, a stereoisomeric mixture of formula (II) is subjected to a bis-decarboxylative bis-amination reaction that comprises a Curtius rearrangement to produce a mixture of stereoisomers of formula (I). As used herein, “Curtius rearrangement” generally refers to a reaction, wherein an acylazide group is converted to an amine or an amine derivative through an isocyanate intermediate. Thus, a bis-decarboxylative bis-amination reaction according to the present invention that proceeds through a Curtius rearrangement comprises first converting the two carboxyl groups of the stereoisomers of formula (II) in the stereoisomeric mixture to bis-acyl azides. The bis-acyl azides are not necessarily isolated, but can be formed in situ and subsequently transformed to the bis-amines or bis-amine derivatives.

In another embodiment of the present invention, a stereoisomeric mixture of formula (II) is subjected to a bis-decarboxylative bis-amination reaction that comprises a Hoffmann rearrangement to produce a mixture of stereoisomers of the formula (I). As used herein, “Hoffmann rearrangement” generally refers to a reaction, wherein an amide group is converted to the corresponding amine or amine derivative via an isocyanate intermediate. Thus, a bis-decarboxylative bis-amination reaction according to the present invention that proceeds through a Hoffmann rearrangement comprises first converting the two carboxyl groups of the stereoisomers of formula (II) in the stereoisomeric mixture to amides.

In yet another embodiment of the present invention, a stereoisomeric mixture of formula (II) is subjected to a bis-decarboxylative bis-amination reaction that comprises a Lossen rearrangement to produce a mixture of stereoisomers of the formula (I). As used herein, “Lossen rearrangement” generally refers to a reaction, wherein a hydroxamic acid or hydroxamic acid derivative is converted to an amine or amine derivative via an isocyanate intermediate. Thus, a bis-decarboxylative bis-amination reaction according to the present invention that proceeds through a Lossen rearrangement comprises first converting the two carboxyl groups of the stereoisomers of formula (II) in the stereoisomeric mixture to two hydroxamic acids or derivatives thereof.

A Curtius rearrangement, Hofmann rearrangement, and Lossen rearrangement all proceed through an isocyanate intermediate. Thus, according to embodiments of the present invention, a method for producing a mixture of stereoisomers of formula (I) comprises first reacting a stereoisomeric mixture of formula (II) with a reagent or reagents to produce a stereoisomeric mixture of diisocyanate intermediates of formula (II). A stereoisomeric mixture of diisocyanate intermediates of formula (II) has the following formula (II-a):

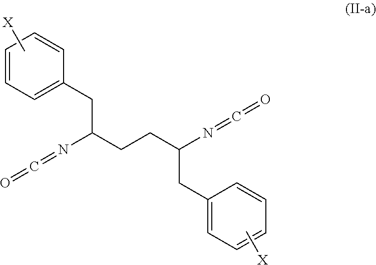

Diisocyanate intermediates (II-a) can be obtained from the corresponding diacyl azide intermediates, dihydroxamic acid intermediates, or diamide intermediates of formula (II). One skilled in the art will recognize that depending upon the particular reaction conditions, diisocyanate intermediates of formula (II-a) can be unstable, and therefore not isolable. Thus, in particular embodiments of the present invention, a stereoisomeric mixture of the diamine compounds of formula (I) can be obtained directly from reacting a stereoisomeric mixture of the corresponding diacyl azide, diamide, or dihydroxamic acid intermediates of formula (II) under Curtius, Hofmann, or Lossen reaction conditions, respectively, without isolating the corresponding diisocyanate intermediates of formula (II-a).

According to an embodiment of the present invention, a stereoisomeric mixture of formula (II) is first reacted with a reagent to convert the carboxyl groups to acyl azides to produce a stereoisomeric mixture of diacyl azide intermediates of formula (II). Any method known in the art for converting carboxyl groups to acyl azides can be used in view of the present disclosure. For example, a stereoisomeric mixture of formula (II) can be reacted with diphenoxyphosphoryl azide (DPPA) to directly convert the carboxyl groups to acyl azides producing diacyl azide intermediates of formula (II). Preferably, the reaction with DPPA is performed in the presence of a base, and more preferably in the presence of triethylamine (NEt₃).

A stereoisomeric mixture of formula (II) can also be reacted with thionyl chloride (SOCl₂) to yield bis-acid chloride intermediates of formula (II), or with an anhydride, acyl halide, or chloroformate ester to yield dianhydride intermediates of formula (II) in a method of the present invention. The bis-acid chloride or dianhydride intermediates of formula (II) can then be treated with an azide reagent, preferably in the presence of a phase-transfer catalyst (PTC), to obtain the diacyl azide intermediates of formula (II).

Examples of acyl halides that can be used in a method of the present invention include, but are not limited to, acetyl chloride. Examples of anhydrides that can be used include, but are not limited to, acetic anhydride and benzoic anhydride. Examples of chloroformate esters that can be used include, but are not limited to ethyl chloroformate. Non-limiting examples of azide reagents include alkali azides, such as sodium azide (NaN₃).

As used herein, “phase-transfer catalyst,” or “PTC,” refer to a reagent that facilitates migration of a reactant from one phase (i.e. solid, liquid, gas) into a second phase, the second phase being the phase in which the desired reaction occurs. For example, a PTC can be used with a reactant that is soluble in an aqueous liquid phase, but not an organic liquid phase, to solubilize the reactant in the organic phase where the desired reaction subsequently takes place. Examples of PTCs that can be used with the present invention, include, but are not limited to, quaternary ammonium and phosphonium salts, such as benzyltrimethylammonium chloride and crown ethers, such as 18-crown-6.

According to embodiments of the present invention, reacting a stereoisomeric mixture of formula (II) under conditions to produce the diacyl azide intermediates of formula (II) is carried out in the presence of a solvent, and preferably an organic solvent, such as toluene and the like. In a preferred embodiment, the solvent is toluene.

According to a preferred embodiment, diacyl azide intermediates of formula (II) are produced by reacting a stereoisomeric mixture of formula (II) with thionyl chloride and sodium azide in the presence of a phase transfer catalyst in toluene.

According to a more preferred embodiment, diacyl azide intermediates of formula (II) are directly produced by reacting a stereoisomeric mixture of formula (II) with DPPA and triethylamine in toluene. Other reaction parameters, such as reaction time and reaction temperature, can vary depending on the particular substrate, solvents, reagents, etc., and one skilled in the art will readily be able to determine the appropriate reaction parameters to achieve the desired results in view of the present disclosure.

According to embodiments of the present invention, diacyl azide intermediates of formula (II) are then exposed to heat for converting to the isocyanate intermediates of formula (II-a), according to a method of the present invention. The heating step can be performed at an elevated temperature, preferably about 50° C., but can vary depending on the substrate. In a particular embodiment, the heating step is performed in the presence of water, such that the diacyl azide intermediates of formula (II) are directly converted to the diamine compounds of formula (I), without isolation of the isocyanate intermediates of formula (II-a).

In another embodiment of the present invention, a stereoisomeric mixture of formula (II) is first reacted with a reagent to convert the carboxyl groups to hydroxamic acids to produce a stereoisomeric mixture of dihydroxamic acid intermediates of formula (II). Methods are well known to those skilled in the art for converting carboxyl groups to hydroxamic acids. For example, carboxyl groups can be converted to esters, such as methyl or ethyl esters, and the corresponding esters can then be treated with hydroxylamine (NH₂OH) under conditions that promote formation of dihydroxamic acids. One of ordinary skill in the art will be able to readily determine the appropriate reaction conditions, i.e., temperature, reagents, solvents, reaction time, etc., for synthesizing a stereoisomeric mixture of dihydroxamic acid intermediates of formula (II) from a stereoisomeric mixture of formula (II).

The dihydroxamic acid intermediates of formula (II) can then be reacted with an acylating agent to produce the corresponding O-acyl or O-aryl hydroxamate intermediates of formula (II). The terms “O-acyl” and “O-aryl” hydroxamate refer to a hydroxamic acid derivative wherein the oxygen atom of the hydroxamic acid functionality is linked to a CO-alkyl or CO-aryl group, respectively. Examples of acylating agents that can be used to convert hydroxamic acids to O-acyl or O-aryl hydroxamates include anhydrides, such as acetic anhydride and benzoic anhydride; acyl halides, such as acetyl chloride; thionyl chloride; and activated aromatic halides, such as dinitrochlorobenzene.

The O-acyl or O-aryl hydroxamate intermediates of formula (II) can then be heated, or treated with base, to yield the corresponding diisocyanate intermediates of formula (II-a). Non-limiting examples of bases that can be used include sodium hydroxide, potassium hydroxide, and diisopropylethylamine. In a particular embodiment, the O-acyl or O-aryl hydroxamate intermediates are treated with heat or base in the presence of water, such that the O-acyl or O-aryl hydroxamate intermediates of formula (II) are directly converted to diamine compounds of formula (I), without isolation of the isocyanate intermediates of formula (II-a).

In yet another embodiment of the present invention, a stereoisomeric mixture of formula (II) is first reacted with a reagent to convert the carboxyl groups to amides to produce a stereoisomeric mixture of diamide intermediates of formula (II). Methods are well known to those skilled in the art for converting carboxyl groups to amides. For example, carboxyl groups can be converted to acid chlorides by reacting with thionyl chloride, followed by treatment of the acid chlorides with ammonia, or other amine reagent such as methylamine, to produce the corresponding amides. The carboxyl groups can also be acylated to produce the corresponding esters or anhydrides, which can then also be converted to the corresponding amines by reacting with ammonia, or other amine reagent such as methylamine. Any acylating reagent in view of the present disclosure can be used. One of ordinary skill in the art will be able to readily determine the appropriate reaction conditions, i.e., temperature, reagents, solvents, reaction time, etc., for synthesizing a stereoisomeric mixture of diamide intermediates of formula (II) from a stereoisomeric mixture of formula (II).

According to embodiments of the present invention, the diamide intermediates of formula (II) are then converted to diisocyanate intermediates of formula (II-a). The diamide intermediates can be reacted with a metal hydroxide and alkali hypobromite, such as sodium hypobromite (NaBrO₂), or alkali hypochlorite, such as sodium hypochlorite (NaClO₂), under basic conditions to promote formation of an N-halogen substituted amide, which then rapidly rearranges to an isocyanate to produce the corresponding diisocyanate intermediates of formula (II-a). Non-limiting examples of metal hydroxides that can be used include sodium hydroxide, potassium hydroxide, barium hydroxide, and calcium hydroxide. In a particular embodiment, the reaction of diamide intermediates with a metal hydroxide and alkali hypobromite or alkali hypochlorite is performed in the presence of water, such that the diamide intermediates of formula (II) are directly converted to a stereoisomeric mixture of diamine compounds of formula (I), without isolation of the isocyanate intermediates of formula (II-a).

According to embodiments of the present invention, when diisocyanate intermediates of formula (II-a) are isolated, a stereoisomeric mixture of diisocyanate intermediates can then be converted to a stereoisomeric mixture of diamine compounds of formula (I) by first reacting the diisocyanate intermediates with a Group I A or Group IIA metal hydroxide to produce metal dicarbamates. Examples of Group IA metal hydroxides that can be used in a method of the present invention include, but are not limited to, lithium hydroxide (LiOH), sodium hydroxide (NaOH), and potassium hydroxide (KOH). Examples of Group IIA metal hydroxides that can be used include, but are not limited to, magnesium hydroxide (Mg(OH)₂) and calcium hydroxide (Ca(OH)₂). In a preferred embodiment, the metal hydroxide is a Group IA metal hydroxide, and is more preferably LiOH. The metal dicarbamates can then be treated with an acid to produce dicarbamic acids, which subsequently lose carbon dioxide, yielding the mixture of stereoisomers of formula (I). Examples of acids that can be used in a method of the present invention include, but are not limited to hydrochloric acid. Preferably, the acid is hydrochloric acid.

In a particularly preferred embodiment, a method of the present invention for producing a mixture of stereoisomers of formula (I) comprises subjecting a stereoisomeric mixture of formula (II) to a bis-decarboxylative bis-amination reaction under Curtius reaction conditions. According to embodiments of the present invention, Curtius reaction conditions comprise converting a stereoisomeric mixture of formula (II) to the corresponding diacyl azide intermediates of formula (II), followed by conversion to the diisocyanate intermediates of formula (II-a). The diisocyanate intermediates of formula (II-a) are then treated to obtain the diamine compounds of formula (I). In one preferred embodiment, Curtius reaction conditions comprise treating a stereoisomeric mixture of formula (II) with DPPA and NEt₃ to produce the corresponding diacyl azide intermediates of formula (II), followed by a heat treatment step to yield the diisocyanate intermediates of formula (II-a), which are then treated with a Group IA or Group IIA metal hydroxide, preferably LiOH, to yield the stereoisomeric mixture of formula (I). In another preferred embodiment, Curtius reaction conditions according to the invention comprise treating a stereoisomeric mixture of formula (II) with SOCl₂, NaN₃, and a phase-transfer catalyst to produce the corresponding diacyl azide intermediates of formula (II), followed by a heat treatment step to yield the diisocyanate intermediates of formula (II-a), which are then treated with a Group IA or Group IIA metal hydroxide, preferably LiOH, to yield the stereoisomeric mixture of formula (I).

Methods for isolating a stereoisomeric mixture of formula (I) from the reaction mixture will be well known to those skilled in the art. For example, conventional techniques, such as extraction, crystallization, filtration, evaporation etc. can be used. Preferably, extraction techniques are used to isolate the stereoisomeric mixture of formula (I) from the reaction mixture. As an illustrative and non-limiting example, the reaction mixture can be separated into aqueous and organic phases, and the organic phase extracted with an acid, such as hydrochloric acid (HCl). The aqueous layers from the extraction and separation of the reaction mixture can be combined, and the combined aqueous layers further extracted with an ether, such as methyl tert-butyl ether (MTBE). The pH of the extracted aqueous layers can then be appropriately adjusted, such that stereoisomers of formula (I) will partition into the organic phase, followed by extraction with an ether, such as MTBE. The organic layers from extraction can be dried over a solid drying agent of an inorganic salt, such as sodium sulfate.

A mixture of stereoisomers of formula (I) produced according to a method of the present invention comprises the (2R,5R)-, (2S,5S)- and meso configurations of formula (I) in a range of ratios of (2R,5R):(2S,5S):meso, wherein the (2R,5R), (2S,5S), and meso configurations of formula (I) can each independently vary from 0.01 to 99.98% (w/w), and a total of (2R,5R)-, (2S,5S)- and meso configurations of formula (I) in the mixture is 100%. For example, the amount of each stereoisomer in the mixture can independently vary, such that it is present in the mixture from 0.01% to a total of 100%, by weight, such as 0.01%, 1%, 5%, 10%, 20%, 30%, 40%, 50%, 60%, 70%, 80%, 90%, or 99% relative to a total of 100%, by weight.

According to embodiments of the present invention a range of ratios of (2R,5R):(2S,5S):meso configurations of formula (I) in a stereoisomeric mixture can range from about 0.01%:0.01%:99.8% to 0.01%:99.8%:0.01% to 99.8%:0.01%:0.01%, such as, for example, 10%:10%:80%, 20%:20%:60%, 25%:25%:50%, 30%:30%:40%, 40%:40%:20%, and 45%:45%:10%. Preferably, a stereoisomeric mixture of formula (I) comprises the (2R,5R)-, (2S,5S)-, and meso configurations in ratio of 25%:25%:50%.

According to a preferred embodiment, a method of the present invention for producing a mixture of stereoisomers of formula (I) produces a stereoisomeric mixture of 1,6-diphenylhexane-2,5-diamine.

According to embodiments of the present invention, the stereoisomeric mixture of formula (II), from which a mixture of stereoisomers of formula (I) is produced, can be obtained using methods known in the art in view of the present disclosure, for example, via hydrolysis of a suitable ester.

In one embodiment, a stereoisomeric mixture of formula (II) can be produced according to the following Scheme (1):

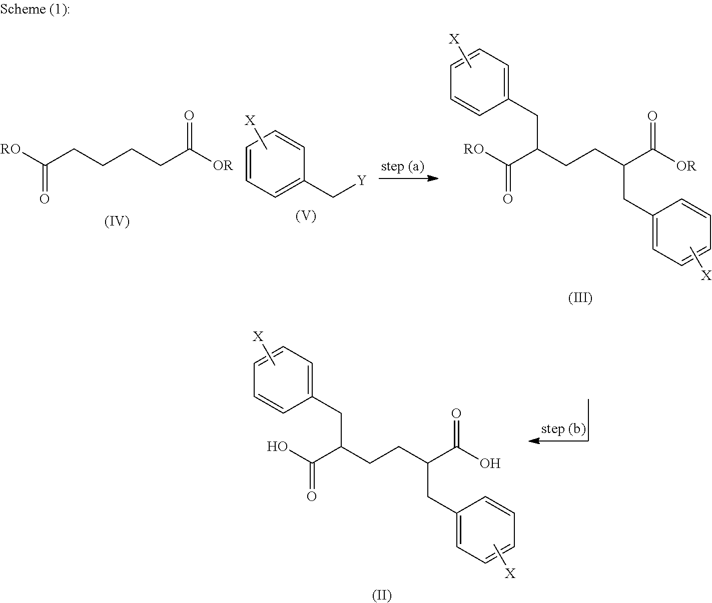

In step (a), an adipic acid ester of formula (IV), wherein R represents an aryl or alkyl group, can be reacted with a benzyl alkylating agent of formula (V) that is optionally substituted with a substituent X selected from the group consisting of halogen, cyano, nitro, alkyl, aryl, alkoxy, polyfluoroalkyl, and polyfluoroaryl, wherein Y represents a chlorine, bromine, iodine, mesylate, tosylate or other suitable leaving group, to produce an ester compound of formula (III). Step (a) can be performed in an organic solvent in the presence of a base. Non-limiting examples of organic solvents for use in step (a) include tetrahydrofuran (THF), and examples of suitable bases include sodium methoxide and sodium ethoxide, preferably sodium ethoxide.

The ester compound of formula (III) is then treated under basic conditions in step (b) to yield a stereoisomeric mixture of formula (II). As an illustrative and non-limiting example, the ester compound of formula (III) can be treated with potassium hydroxide in the presence of methanol and water to yield a stereoisomeric mixture of formula (II).

According to a preferred embodiment, the adipic acid ester of formula (IV) is diethyl hexanedioic acid (R=ethyl). According to another preferred embodiment, the benzyl alkylating agent of formula (V) is not substituted with a substituent X. In a most preferred embodiment, the benzyl alkylating agent of formula (V) is benzyl chloride.

In a particularly preferred embodiment, a stereoisomeric mixture of formula (I) is produced according to a method of the present invention as shown in the following Scheme (2):

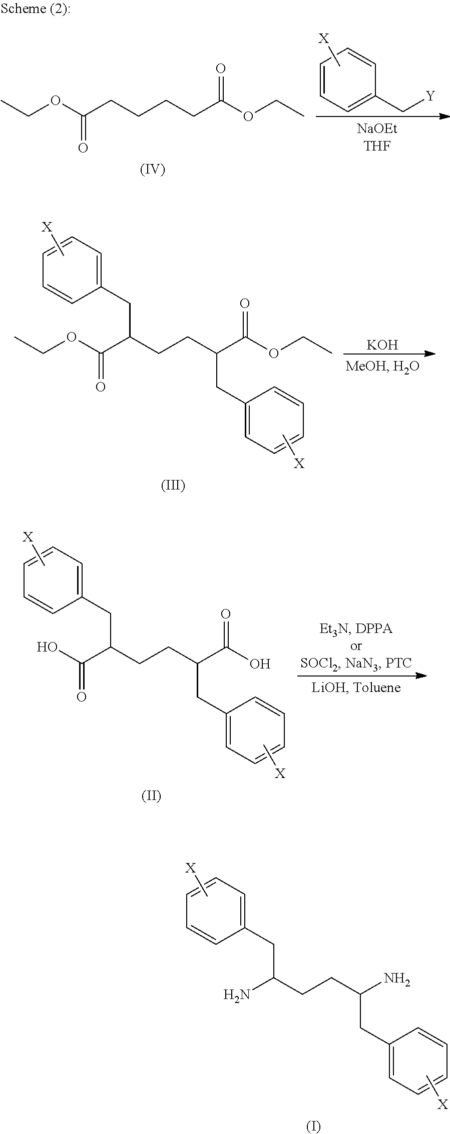

wherein the aryl groups of formula (I) are optionally substituted with a substituent X selected from a group consisting of halogen, cyano, nitro, alkyl, aryl, alkoxy, polyfluoroalkyl and polyfluoroaryl, wherein the X substituents are identical and are both located at either the para, meta, or ortho positions.

According to an embodiment of the present invention as shown in Scheme (2), the synthesis of an ester compound of formula (III) is afforded by adding diethyl hexanedioic acid (IV) to a solution of sodium ethoxide (NaOEt) in tetrahydrofuran (THF). To that solution is added a suitable benzyl alkylating agent that is optionally substituted with a substituent X selected from the group consisting of halogen, cyano, nitro, alkyl, aryl, alkoxy, polyfluoroalkyl, and polyfluoroaryl, wherein Y is chlorine, bromine, or iodine. Preferably, the benzyl alkylating reagent is benzyl chloride. A suitable alcohol is added and the reaction is neutralized with acid. The reaction mixture is separated into organic and aqueous phases, and the aqueous phase extracted using an organic solvent. The organic layers are collected and concentrated to generate the ester compound of formula (III).

The synthesis of a stereoisomeric mixture of formula (II) can then be afforded via an acidification step, by adding water and potassium hydroxide (KOH) to a solution of the ester compound (III) in methanol, followed by heating to reflux. After removing the solvent, methyl tert-butyl ether (MTBE) and water are added. The aqueous layer is collected and treated with acid. The resulting precipitate is collected, washed with water, and dried to produce a stereoisomeric mixture of the diacid of formula (II). The synthesis of a stereoisomeric mixture of the diamine of formula (I) can then be achieved via an acidification step, by dissolving the stereoisomeric mixture of formula (II) and triethylamine (Et₃N) in toluene, forming a precipitate. After filtering the resulting precipitate, the filtrate is treated with diphenoxyphosphoryl azide (DPPA) under Curtius reaction conditions. The reaction mixture is then transferred to an addition funnel and added to a solution of lithium hydroxide (LiOH) in water. After quenching the reaction by adding to cooled acid, the mixture is separated and the organic phase extracted with acid. The aqueous portion is then back extracted with MTBE. The combined organic layers are treated with an aqueous caustic solution and extracted with MTBE. The organic layer is collected, dried over sodium sulfate and concentrated to afford a stereoisomeric mixture of the diamine of formula (I).

In another general aspect, the present invention relates to a method of preparing a stereoisomerically pure compound of formula (Ia) of the (2R,5R) configuration:

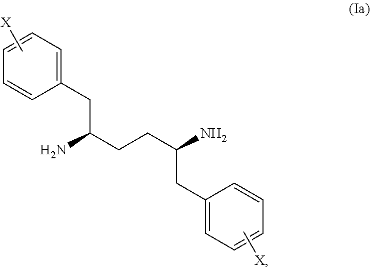

or a pharmaceutically acceptable salt thereof;
a stereoisomerically pure compound of formula (1b) of the (2S,5S) configuration:

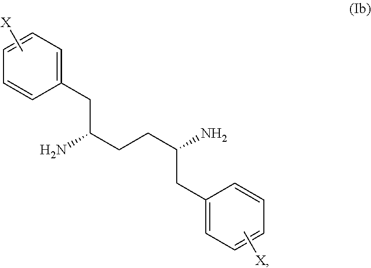

or a pharmaceutically acceptable salt thereof; or
a stereoisomerically pure compound of formula (Ic) of the meso configuration:

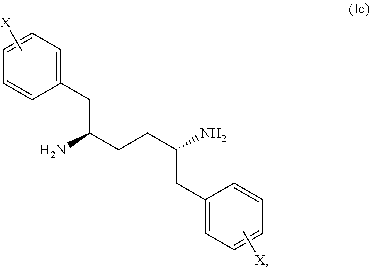

or a pharmaceutically acceptable salt thereof,
the method comprising producing a mixture of stereoisomers of formula (I):

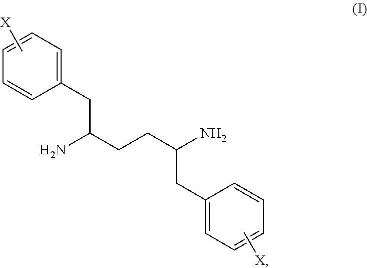

or pharmaceutically acceptable salts thereof, wherein the mixture comprises the (2R,5R)-, (2S,5S)-, and meso configurations, and isolating the stereochemically pure compound of formula (Ia), (Ib), or (Ic), or pharmaceutically acceptable salt thereof from the mixture of stereoisomers. In the mixture, the (2R,5R), (2S,5S), and meso are present in a range of ratios. For example, each of the stereoisomers can independently vary from 0.01 to 99.98% (w/w), such that a total of (2R,5R), (2S,5S), and meso configurations of formula (I) in the mixture is 100%. Each aryl group of formula (I) is optionally independently substituted with a substituent X selected from the group consisting of halogen, cyano, nitro, alkyl, aryl, alkoxy, polyfluoroalkyl and polyfluoroaryl. Preferably, the X substituents are identical and are located at identical locations, either the para, meta, or ortho positions.

According to embodiments of the present invention, the mixture of stereoisomers of formula (I) is produced by a method according to an embodiment of the present invention, for example, by subjecting a mixture of stereoisomers of formula (II) to a bis-decarboxylative bis-amination reaction. Preferably, the mixture of stereoisomers of formula (I) is produced by subjecting a mixture of stereoisomers of formula (II) to a bis-decarboxylative bis-amination reaction that comprises a Curtius rearrangement.

Any suitable purification or separation technique known to an ordinary person of skill in the art can be utilized to selectively isolate the desired stereoisomer of formula (I) from the stereoisomeric mixture in view of the present disclosure. According to embodiments of the present invention, such method can be based on, for example, kinetic resolution, chromatography and particularly chiral chromatography, or diastereomeric derivatization, recrystallization, and subsequent stereoisomer generation.

According to a preferred embodiment of the present invention, a step of isolating a stereochemically pure compound of formula (Ia), (Ib), or (Ic) from a mixture of stereoisomers comprises chiral chromatography.

As used herein, “chiral chromatography” refers to a column chromatographic separation technique, wherein the stationary phase of the column differentially interacts with different stereoisomers of a particular compound, such that the different stereoisomers can be separated from one another to obtain a stereoisomerically pure compound. Non-limiting examples of column chromatography that can be employed in chiral chromatography according to the invention include batch chromatography either under low pressure, moderate pressure (i.e., flash chromatography), or high pressure (i.e. high performance liquid chromatography, HPLC); supercritical fluid chromatography; and continuous chromatography including multi column chromatography in continuous mode, such as Simulated Moving Bed (SMB) and Varicol, and multi column chromatography in semi continuous mode, such as Steady State Recycling or Multi Column Solvent Gradient Purification (MSCGP) separation techniques.

According to a particularly preferred embodiment of the present invention, a stereochemically pure compound of formula (Ia), (Ib), or (Ic) is isolated from a mixture of stereoisomers using a continuous chromatographic separation technique, such as SMB or Varicol.

According to embodiments of the present invention, chiral chromatography, such as SMB separation techniques, can be performed using a chromatographic media that allows for the resolution, or separation, of the stereoisomers of a compound of formula (I), such that a stereoisomerically pure compound of formula (Ia), (Ib), or (Ic) can be obtained, or each stereoisomer of a compound of formula (I) can be obtained in its stereoisomerically pure form. In one embodiment, the chromatographic media is a silica based media. In another embodiment, the chromatographic media is a ceramic based media. In yet another embodiment, the chromatographic media is a polymer based media. In a particular embodiment, the chromatographic media is a silica based media that has a mono-distributed particle size with an average particle size distribution comprised between about 5-50 micrometers, such as, for example, 5, 10, 16, 20, 30 and 50 micrometers. In yet another embodiment, the chromatographic media comprises a polymer that exhibits preferential selectivity towards one of the stereoisomers. The chromatographic media can be coated with the polymer, or bound to the polymer, such as, for example, via a covalent bond. Thus, in yet another embodiment, the chromatographic media is coated with a functionalized amylose or cellulose based chiral selector. Even further, in another embodiment, the amylose or cellulose based chiral selector is permanently bonded to the chromatographic media. For example, the chromatographic media can be a silica based media with an amylose or cellulose based chiral selector bonded to the silica based media.

In one embodiment, the chromatographic media comprises the chiral selector cellulose tris(4-methylbenzoate), such as, for example, Chiralcel® OJ manufactured by Daicel (Japan). In another embodiment, the chromatographic media comprises the chiral selector amylose tris(3,5-dimethylphenylcarbamate), such as, for example, Chiralpak® AD manufactured by Daicel (Japan). In yet another embodiment, the chromatographic media comprises the chiral selector amylose tris(3,5-dimethylphenylcarbamate), such as, for example, Chiralpak® IA manufactured by Daicel (Japan).

According to embodiments of the present invention, the chromatographic separation can be performed using a mobile phase. In one embodiment, the mobile phase comprises a single organic solvent, a mixture of organic solvents, or a mixture of one or more organic solvents, water and liquid or supercritical carbon dioxide. Examples of organic solvents that can be used for the mobile phase include, but are not limited to, acetonitrile, acetone, methanol, ethanol, isopropanol, n-propanol, n-butanol, tert-butanol, hexanes (e.g., n-hexane), heptanes (e.g., n-heptane), cyclopentatnes, methylcyclopentanes, cyclohexanes, methylcyclohexanes, methylcycloheptanes, ethylacetate, methyl-tert-butyl ether, toluene, dichloromethane, trichloroethane, trichloromethane, dichloroethane, and mixtures thereof. In a preferred embodiment, the mobile phase is comprised of a mixture of a hydrocarbon and an alcohol, such as a mixture of n-heptane and either isopropyl alcohol or ethanol in variable or volumetric ratio. The percentages of each solvent in a mixture of solvents can vary over a wide range, and one skilled in the art will readily be able to determine the appropriate amount of each solvent in a mixture of solvents in view of the present disclosure.

In another preferred embodiment, a method for isolating a stereochemically pure compound of formula (I) from a mixture of stereoisomers of formula (I) further comprises recycling of the unwanted stereoisomer(s) of formula (I) through a racemization process. For example, the (2S,5S)- and meso compounds of a 1,6-diaryl-2,5-diaminohexane according to the invention can be recycled through a racemization process followed by optical purification to the (2R,5R)-stereoisomer. As used herein, the term “racemization” refers to a process of converting a stereoisomerically pure compound into a stereoisomeric mixture, wherein more than one stereoisomer of the compound is present in the mixture. Methods of racemization of the unwanted stereoisomeric amines, such as that described by Escoubet et al., J. Org Chem. 2006, 71, 7288-7292, can be modified for use in the present invention in view of the present disclosure. For example, (2S,5S) and (2R,5R) configurations of formula (I) can be racemized by refluxing in an organic solvent, such as benzene, in the presence of a radical compound, such as azobisisobutyronitrile (AIBN) to convert each of the stereoisomerically pure compounds into a stereoisomeric mixture of formula (I). See, e.g., Example 4 below.

According to embodiments of the present invention, a stereoisomeric mixture of formula (I) obtained from racemization of a stereoisomerically pure compound of formula (Ia), (Ib), or (Ic) can be subjected to a purification or separation technique to obtain a stereoisomerically pure compound of formula (Ia), (Ib), or (Ic). Any method for purifying or separating the stereoisomers from a stereoisomeric mixture can be used in view of the present disclosure. Preferably, a continuous chromatographic technique such as SMB is used. The number of times a stereoisomerically pure compound of the present invention can be racemized to obtain a stereoisomeric mixture that is then further purified or separated to obtain a stereoisomerically pure compound is not limited in anyway. In this way, the maximum yield of the desired stereoisomer can be obtained.

In yet another general aspect, the present invention relates to a mixture of stereoisomers of formula (I):

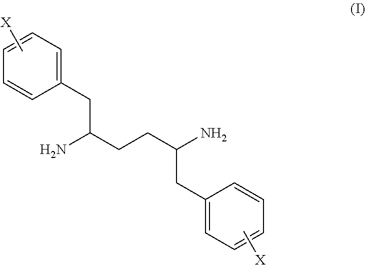

comprising (2R,5R)-, (2S,5S)-, and meso configurations of formula (I). The mixture can have a range of ratios of (2R,5R):(2S,5S):meso configurations of formula (I). The (2R,5R), (2S,5S), and meso configurations of formula (I) can each independently vary, e.g., from 0.01 to 99.98% (w/w), such that a total of the (2R,5R), (2S,5S), and meso configurations of formula (I) in the mixture is 100%. Each aryl group of formula (I) is optionally substituted with a substituent X selected from the group consisting of halogen, cyano, nitro, alkyl, aryl, alkoxy, polyfluoroalkyl and polyfluoroaryl, preferably the X substituents are identical and are located at the same locations, either the para, meta, or ortho positions, in the two aryl groups. For example, the mixture of stereoisomers of formula (I) can comprise the (2R,5R)-, (2S,5S)-, and meso configurations of formula (I) in a ratio of (2R,5R):(2S,5S):meso of 25%:25%:50%.

In a preferred embodiment, the present invention relates to a mixture of stereoisomers of 1,6-diphenylhexane-2,5-diamine. The mixture comprises (2R,5R)-1,6-diphenylhexane-2,5-diamine, (2S,5S)-1,6-diphenylhexane-2,5-diamine, and meso-1,6-diphenylhexane-2,5-diamine, preferably in a ratio (2R,5R):(2S,5S):meso of 25%:25%:50%.

The mixture of stereoisomers according to embodiments of the present invention can be used as reagents in a reaction. It can also be used as a source for the production of enantiomerically pure diamines of formula (Ia) or (Ib), which can be further used in chiral reactions, e.g., for the production of a pharmaceutically active ingredients, including, but not limited to cobicistat.

In a preferred embodiment, the present invention relates to a mixture of stereoisomers of 1,6-diphenylhexane-2,5-diamine produced by a method according to an embodiment of the present invention.

Without limiting uses, the chemical composition herein may be used as a chiral auxiliary, ligand, or catalyst.

This invention will be better understood by reference to the non-limiting example that follows, but those skilled in the art will readily appreciate that the example is only illustrative of the invention as described more fully in the claims which follow thereafter.

### EXAMPLES

The following abbreviations will be used in the Examples unless stated otherwise:

NaOEt: sodium ethoxide

THF: tetrahydrofuran

HCl: hydrochloric acid

N₂: nitrogen

NMR: nuclear magnetic resonance spectroscopy

MS: mass spectroscopy

EI-MS: electrospray ionization mass spectrometry

MeOH: methanol

MTBE: Methyl tert-butyl ether

KOH: potassium hydroxide

Et₃N: triethylamine

LiOH: lithium hydroxide

AIBN: azobisisobutyronitrile

HPLC: high performance liquid chromatography

### Example 1

### Synthesis of diethyl-2,5-dibenzylhexanedioic acid

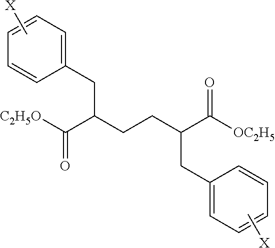

In a 20 L jacketed reactor, NaOEt (632 g) was suspended in 5200 mL THF and heated to 60° C. Diethyl hexanedioic acid (1520 g) was added slowly, maintaining an internal temperature≦65° C. The approximate addition time was 50 min. The mixture was stirred for 16 h at 60° C. Benzyl chloride (1066 g) was added over 3.75 h. The mixture was stirred at 60° C. for 3.5 h then cooled to 22° C. Additional NaOEt (635 g) was added over 2 h, heated to 60° C., and stirred for 16 h. Benzyl chloride (1143 g) was added over 1.75 h. The mixture was heated to 65° C. for 6 h and then cooled to 30° C. NaOEt (130 g) was added and the mixture heated to 60° C. for 5 h to convert the ring closed cyclopentanone intermediate to the desired open chain form. Benzyl chloride (269 g) was added over 1.75 h and the mixture stirred at 60° C. for 2 h to convert any unreacted NaOEt to benzyl ethyl ether. Ethanol (291 g) was added and the mixture was cooled to 25° C. To the solution was added 4775 mL of 0.01 M HCl to neutralize the bases. Toluene (4 L) was added, the organic layer was collected, and the organic liquid was concentrated. The resulting oil (3361 g, 95.4%) was further dried with a N₂ sweep to remove volatiles.

The equilibrium mixture of stereoisomers was characterized by ¹H NMR, ¹³C NMR, and MS. ¹H NMR (300 MHz, CDCl₃) δ 7.30-7.13 (m, 10H), 4.08-3.98 (m, 4H), 2.97-2.88 (m, 2H), 2.77-2.60 (m, 4H), 1.75-1.62 (m, 2H), 1.57-1.46 (m, 2H), 1.36-1.23 1.13 (ap t, J=7.1 Hz, 6H); ¹³C NMR (75 MHz, CDCl₃) δ 175.0, 139.0, 128.8, 128.2, 162.2, 60.1, 47.1, 38.3, 29.3, 14.1; EI MS m/z 382 (M)⁺.

### Example 2

### Synthesis of 2,5-dibenzylhexanedioic acid

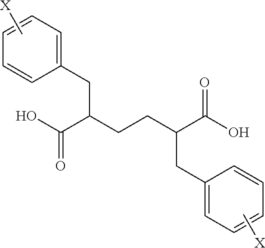

In a jacketed reactor, 500 g of diethyl-2,5-dibenzylhexanedioic acid (DBA) was suspended in 650 mL of MeOH. Water was charged (150 mL) followed by addition of solid KOH (220 g). The approximate addition time was 20 min, such that the internal temperature was ≦60° C. After the addition was complete, the system was fitted with a reflux condenser and heated to 60° C. for 12 h. Upon reaction completion, the reflux condenser was replaced with a distillation head. Solvent was stripped under house vacuum (15-20 in Hg) to yield 500 mL of a thick slurry. MTBE (500 mL) and water (500 mL) were charged, the mixture stirred, and was allowed to settle. The aqueous layer was transferred to a jacketed reactor and cooled to 0° C. 37% HCl (390 g) was charged slowly over 50 min, maintaining an internal temperature of ≦10° C., to form a solid precipitate. The solid was filtered, washed with water (300 mL), and dried. 2,5-dibenzyladipic acid was isolated as a white solid (336 g, 78.7%).

The equilibrium mixture of stereoisomers was characterized by ¹H NMR, ¹³C NMR, and MS. ¹H NMR (400 MHz, DMSO-d₆) δ 12.2 (s, 2H), 7.25-7.15 (m, 10H), 2.84-2.74 (m, 2H), 2.70-2.62 (m, 2H), 2.58-2.46 (m, 2H), 1.60-1.40 (m, 4H); ¹³C NMR (100 MHz, CDCl₃) δ 176.5, 139.9, 129.24, 128.7, 126.5, 47.1, 38.0, 29.8; EI MS m/z 326 (M)⁺.

### Example 3

### Synthesis of 1,6-diphenylhexane-2,5-diamine

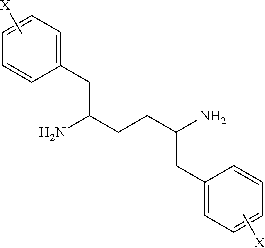

In a jacketed reactor, 2,5-dibenzylhexanedioic acid (225 g) and Et₃N (147 g) were dissolved in 1.5 L of toluene. A minimal precipitate was observed and the mixture was filtered to remove the solids. The filtrate was returned to the reactor and cooled to 5° C. DPPA (398 g) was charged over 20 min. A moderate exotherm was observed (internal temperature≦15° C.). The mixture was stirred at 5° C. for 6 h, warmed to 22° C., and held for 12 h (minimal bubbling was observed). The mixture was heated to 50° C., held for 3 h, and returned to 22° C. This diisocyanate mixture was transferred to an addition funnel. To the reactor was charged LiOH (145 g) and water (2 L). This solution was cooled to 5° C. whereupon the diisocyanate mixture was added at a rate of 25 mL/min. Upon complete addition, the mixture was warmed to 22° C. where a white precipitate formed (Li dicarbamate). To a separate reactor, 37% HCl (900 g) was charged and cooled to 5° C. The carbamate slurry was slowly charged such that the internal temperature was ≦20° C. The mixture stirred for 2 h and was allowed to settle. The aqueous phase was separated and the organic phase extracted with 2 N HCl (3×1.5 L). The combined aqueous layers were back extracted with MTBE (1 L) and then cooled to 5° C. A 30% aqueous caustic solution (1.2 kg) was charged until a pH of 12 was reached. This suspension was extracted with MTBE (3×1.5 L), dried over sodium sulfate (300 g), and concentrated to deliver an amorphous white solid (140 g, 75.8%).

The mixture of stereoisomers was characterized by ¹H NMR, ¹³C NMR, and HPLC. ¹H NMR (300 MHz, CDCl₃) δ 7.31-7.16, (m, 10H), 3.01-2.93 (m, 2H), 2.83-2.76 (m, 2H), 2.50-2.41 (m, 2H), 1.66-1.36 (m, 4H), 1.12 (broad s, 4H); ¹³C NMR (75 MHz, CDCl₃) δ 139.3, 129.1, 128.2, 126.0, 52.7, 44.6, 34.1.

### Example 4

### Racemization of (2S,5S)- and (2R,5R)-1,6-diphenylhexane-2,5-diamine

In a 125 mL jacketed reactor, (2S,5S)-, (2R,5S)-, (2S,5R)- or (2R,5R)-1,6-diphenylhexane-2,5-diamine (1.0 g) and octane thiol (0.65 g) were dissolved in benzene (70 mL). A condenser was attached and the mixture heated to reflux using an 85° C. jacket temperature. AIBN (0.12 g) was charged in three equal portions over 1.5 h. After complete addition, this mixture was stirred at reflux for 12 h. After chiral HPLC indicated the mixture was fully epimerized, the mixture was cooled to 22° C. The organic mixture was extracted with 2N HCl. The combined aqueous extracts were washed with MTBE and subsequently basified with caustic. The basified aqueous portion was further extracted with MTBE. The combined organic extracts were dried with sodium sulfate and concentrated to dryness delivering a yellow oil (1.0 g, 100%). The resulting mixture of stereoisomers was characterized by chiral HPLC comparison to that of an authentic sample.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of producing a mixture of stereoisomers of formula (I): 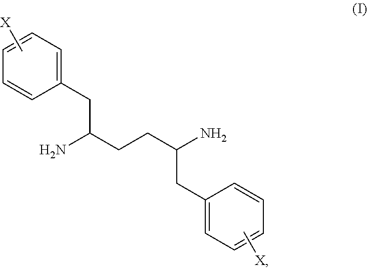
or pharmaceutically acceptable salts thereof,
comprising subjecting a stereoisomeric mixture of formula (II): 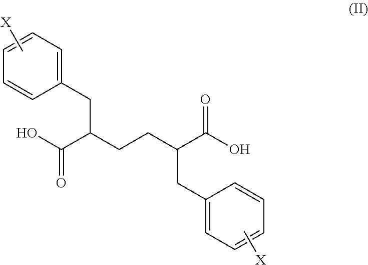
or pharmaceutically acceptable salts thereof to a bis-decarboxylative bis-amination reaction, wherein the mixture of stereoisomers of formula (I) comprises (2R,5R)-, (2S,5S)- and meso configurations of formula (I), and X is independently selected from the group consisting of hydrogen, halogen, cyano, nitro, alkyl, aryl, alkoxy, polyfluoroalkyl and polyfluoroaryl.

2. The method of claim 1, wherein the bis-decarboxylative bis-amination reaction comprises a Curtius Rearrangement.

3. The method of claim 1, wherein the bis-decarboxylative bis-amination reaction comprises a Hoffmann Rearrangement.

4. The method of claim 1, wherein the bis-decarboxylative bis-amination reaction comprises a Lossen Rearrangement.

5. The method of claim 1, wherein the bis-decarboxylative bis-amination reaction comprises treating the stereoisomeric mixture of formula (II) with diphenoxyphosphoryl azide (DPPA) and a Group IA or Group IIA metal hydroxide.

6. The method of claim 1, wherein each of the aryl groups in formula (I) and formula (II) has an identical X substituents located at an identical para-, meta-, or ortho-position.

7. The method of claim 1, wherein the X is hydrogen and the mixture of stereoisomers of formula (I) comprises (2R,5R)-1,6-diphenyl-2,5-diaminohexane, (2S,5S)-1,6-diphenyl-2,5-diaminohexane, and meso-1,6-diphenyl-2,5-diaminohexane.

8. A method of preparing
a stereoisomerically pure compound of formula (Ia) of the (2R,5R) configuration: 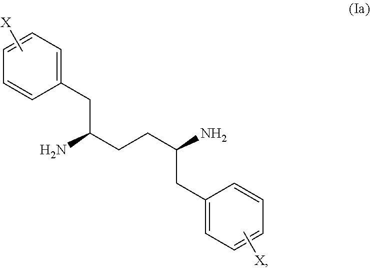
or a pharmaceutically acceptable salt thereof;
a stereoisomerically pure compound of formula (1b) of the (2S,5S) configuration: 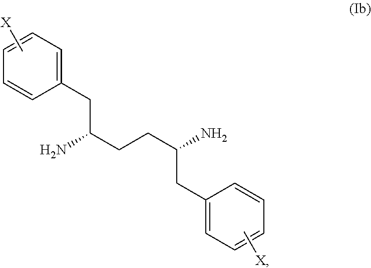
or a pharmaceutically acceptable salt thereof; or
a stereoisomerically pure compound of formula (Ic) of the meso configuration: 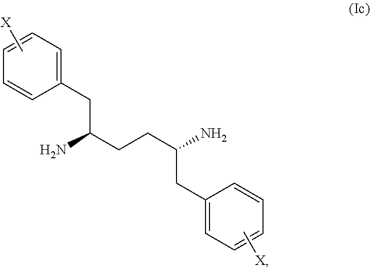
or a pharmaceutically acceptable salt thereof;
comprising:
(i) producing a mixture of stereoisomers of formula (I): 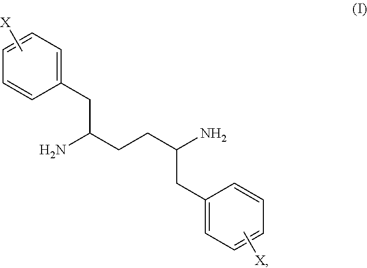
or pharmaceutically acceptable salts thereof, wherein the mixture comprises (2R,5R)-, (2S,5S)-, and meso configurations of formula (I), and X is independently selected from the group consisting of hydrogen, halogen, cyano, nitro, alkyl, aryl, alkoxy, polyfluoroalkyl and polyfluoroaryl; and
(ii) isolating the stereoisomerically pure compound of formula (Ia), (Ib), or (Ic), or the pharmaceutically acceptable salt thereof from the mixture of stereoisomers.

9. The method of claim 8, wherein the isolating step comprises chiral chromatography.

10. The method of claim 9, wherein the chiral chromatography comprises a continuous chromatographic technique.

11. The method of claim 8, wherein the producing step comprises subjecting a stereoisomeric mixture of formula (II): 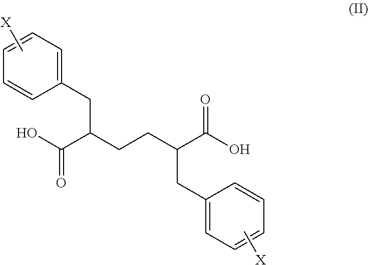
or pharmaceutically acceptable salts thereof to a bis-decarboxylative bis-amination reaction comprising one selected from the group consisting of a Curtius Rearrangement, a Hoffmann Rearrangement and a Lossen Rearrangement.

12. The method of claim 11, wherein the bis-decarboxylative bis-amination reaction comprises treating the stereoisomeric mixture of formula (II) with diphenoxyphosphoryl azide (DPPA) and a Group IA or Group IIA metal hydroxide.

13. The method of claim 8, wherein each of the aryl groups in formula (I) has an identical X substituents located at an identical para-, meta-, or ortho-position.

14. The method of claim 8, wherein the X is hydrogen and the stereoisomerically pure compound of formula (Ia), (Ib), or (Ic) is stereoisomerically pure (2R,5R)-1,6-diphenyl-2,5-diaminohexane, (2S,5S)-1,6-diphenyl-2,5-diaminohexane, or meso-1,6-diphenyl-2,5-diaminohexane, respectively.

15. The method of claim 8, further comprising subjecting at least one of the stereoisomerically pure compound of formula (Ia), (Ib), or (Ic) to a racemization process.

16. A method of preparing a stereoisomerically pure (2R,5R)-1,6-diphenyl-2,5-diaminohexane: 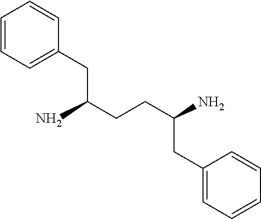 [SMILES: N[C@H](CC[C@@H](N)Cc1ccccc1)Cc1ccccc1]
or a pharmaceutically acceptable salt thereof;
comprising:
(i) producing a mixture of stereoisomers of 1,6-diphenyl-2,5-diaminohexane: 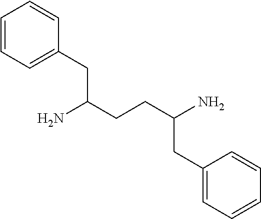 [SMILES: NC(CCC(N)Cc1ccccc1)Cc1ccccc1]
or pharmaceutically acceptable salts thereof, wherein the mixture comprises (2R,5R)-1,6-diphenyl-2,5-diaminohexane, (2S,5S)-1,6-diphenyl-2,5-diaminohexane, and meso-1,6-diphenyl-2,5-diaminohexane; and
(ii) isolating the stereoisomerically pure (2R,5R)-1,6-diphenyl-2,5-diaminohexane or the pharmaceutically acceptable salt thereof from the mixture by a method comprising a simulated moving bed chromatographic technique.

17. The method according to claim 16, wherein the producing step comprises subjecting a stereoisomeric mixture of diethyl-2,5-dibenzylhexanedioic acid: 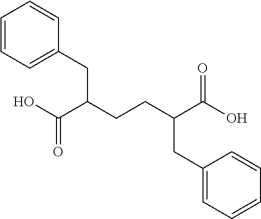 [SMILES: O=C(O)C(CCC(Cc1ccccc1)C(=O)O)Cc1ccccc1]
or pharmaceutically acceptable salts thereof to a bis-decarboxylative bis-amination reaction comprising one selected from the group consisting of a Curtius Rearrangement, a Hoffmann Rearrangement and a Lossen Rearrangement.

18. A mixture of stereoisomers of formula (I): 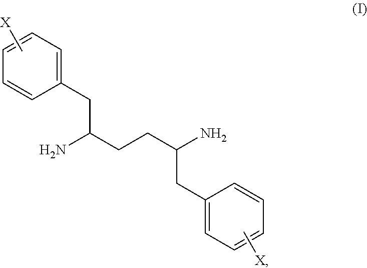
or pharmaceutically acceptable salts thereof, comprising (2R,5R)-, (2S,5S)-, and meso configurations of formula (I), and X is independently selected from the group consisting of hydrogen, halogen, cyano, nitro, alkyl, aryl, alkoxy, polyfluoroalkyl and polyfluoroaryl.

19. The mixture of claim 18, wherein each of the aryl groups in formula (I) has an identical X substituents located at an identical para-, meta-, or ortho-position.

20. The mixture of claim 18, wherein the X is hydrogen and the mixture comprises (2R,5R)-1,6-diphenyl-2,5-diaminohexane, (2S,5S)-1,6-diphenyl-2,5-diaminohexane, and meso-1,6-diphenyl-2,5-diaminohexane.
